(19) United States
(12) Patent Application Publication
(10) Pub. No.: US 2020/0010861 A1
(43) Pub. Date: Jan. 9, 2020

(54) PROCESSES AND BIOREACTORS FOR GAS FERMENTATION PRODUCTS

(71) Applicant: UNIVERSITY OF HAWAI'I, Honolulu, HI (US)

(72) Inventors: Jian YU, Honolulu, HI (US); Pradeep MUNASINGHE, Honolulu, HI (US)

(21) Appl. No.: 16/468,607

(22) PCT Filed: Dec. 6, 2017
(86) PCT No.: PCT/IB2017/057696

§ 371 Date: Jun. 11, 2019

Related U.S. Application Data
(60) Provisional application No. 62/433,748, filed on Dec. 13, 2016.

(51) Int. Cl. C12P 7/62; C12M 1/00; C12M 1/107

(52) U.S. Cl. CPC C12P 7/625; C12M 21/04; C12M 29/18; C12M 29/06

(57) ABSTRACT

A process for producing at least one polyhydroxyalkanoate through gas fermentation may include: providing at least one gas fermentation vessel having a volume partially filled with a liquid fermentation broth that includes: water, suspended gas-fermenting microorganisms capable of producing the at least one polyhydroxyalkanoate, and nutrients for the gas-fermenting microorganisms, a remaining part of the volume of the at least one gas fermentation vessel being filled with a gas phase; continuously withdrawing an aliquot of the liquid fermentation broth from the at least one gas fermentation vessel; supplying a gaseous substrate comprising CO₂, H₂, and O₂; cultivating the gas-fermenting microorganisms to form a cell mass containing the at least one polyhydroxyalkanoate; and recovering the at least one polyhydroxyalkanoate from the cell mass. A gas fermentation bioreactor may include: a vessel, at least one effluent circulation conduit communicating with the vessel interior, at least one spraying nozzle, and a feeding system.

### STATEMENT REGARDING FEDERALLY SPONSORED R&D

This invention was made with government support under N00014-10-1-0310, N00014-11-1-0391, N00014-12-1-0496, N00014-13-1-0463, N00014-14-1-0054, N00014-15-1-0028 and N00014-16-1-2116 awarded by the Office of Naval Research, USA. The government has certain rights in the invention.

### FIELD OF THE INVENTION

The present invention relates to a process for producing gas fermentation products and to a bioreactor that can be used for carrying out the process. The process and bioreactor of the present invention are particularly suitable for producing microbial polyhydroxyalkanoates (PHAs) using a gaseous feedstock containing CO₂.

### BACKGROUND OF THE INVENTION

CO₂ released from fossil fuel combustion is a prime greenhouse gas. A typical flue gas contains (% vol): CO₂ (6-12), O₂ (6-14), N₂ (66-76), H₂O (6-18) and other minor pollutants. With the growing concerns over climate change, reducing CO₂ emission by carbon capture and sequestration becomes a serious challenge to large point sources such as power plants, cement plants and municipal solid waste incinerators. Converting CO₂ into valuable products is an attractive alternative to storage. Hydrogen-oxidizing bacteria such as Cupriavidus necator can fix CO₂ by using H₂ and O₂ in lithoautotrophic conditions. Since H₂ and O₂ can be conveniently obtained from water electrolysis with renewable energy such as solar and wind power, it is a sustainable process to produce biomass from CO₂ through gas fermentation. Importantly, a substantial amount (40-80 wt %) of C. necator cell mass under controlled conditions is a polyhydroxyalkanoate (PHA), specifically polyhydroxybutyrate (PHB). This bio-polyester, which is accumulated in the microbial cells as energy and carbon storage material, can be processed to obtain a variety of valuable products including thermoplastics, high-grade fuels, alkenes and aromatics. Gas fermentation of hydrogen-oxidizing bacteria has been investigated almost exclusively with a mixture of CO₂, H₂ and O₂. To the Applicant's knowledge no research has used flue gas because the large amount of N₂ dilutes the concentrations of gas substrates and makes the bioreactor operation difficult.

Microbial CO₂ fixation is limited by the mass transfer of the gaseous substrates to the microbial cells suspended in an aqueous mineral solution (i.e. fermentation broth). The poor solubility of the gases, H₂ and O₂ in particular, in the fermentation broth poses a great technical challenge to a gas fermentation for high CO₂ fixation rate, high cell density and high PHB productivity. In a conventional aerated bioreactor, the gas is introduced at the bottom and the gas bubbles arise in the aqueous solution quickly. Although the mass transfer around the bubbles can be intensified by mechanical agitation, a high gassing rate is necessary to generate a high gas hold-up or interfacial area in the liquid phase. Most of the gas is therefore discharged and wasted. This problem may be solved by recirculating the gas in a closed bioreactor system. Such a solution, however, is not applicable to a flue gas.

An ideal bioreactor for gas fermentation should have a high gas mass transfer coefficient (k_{L}a) even at a very low gassing rate. In a conventional agitated bioreactor, however, the k_{L}a value declines with decrease of the gassing rate, meaning that a high energy dissipation in liquid phase cannot secure a high k_{L}a at a low gassing rate.

Spray columns have been used in industries for gas absorption, but mainly for the fast reactions in liquid solution such as SO₂ absorption in NaOH solution and CO₂ absorption in amine solution, in which the mass transfer resistance is primarily located at the gas side. In contrast, the mass transfer resistance of CO₂, H₂ and O₂ in microbial fermentation is primarily located at the liquid side and is much higher than the gas-side mass transfer resistance.

To the Applicant's knowledge, however, nozzle spraying has not been used in microbial fermentation.

### SUMMARY OF THE INVENTION

In view of the disadvantages of the prior art, the Applicants have faced the problem of providing a process for producing a gas fermentation product such as polyhydroxyalkanoates (PHAs) and a gas fermentation bioreactor which can be operated maintaining a high gas mass transfer rate even at very low gas flow rate (Uₛ), i.e. gassing rate. The retention time of gas molecules in the bioreactor can therefore be controlled to give a high efficiency utilization.

The Applicants found that the above advantages, and others which will be apparent from the following description, can be achieved by contacting the fermentation broth (medium solution) in the form of sprayed droplets with the gaseous substrate in the overhead gas phase present in the gas fermentation vessel of the bioreactor.

The contact can be achieved in several ways. In a first preferred embodiment, the fermentation broth is withdrawn from the vessel, circulated via an external loop and then re-introduced into the bioreactor by spraying it into the overhead gas phase present therein, where it comes in contact with the gaseous substrate which is separately supplied into the gas phase of the bioreactor. The fermentation broth can be driven in the external loop for example by a positive replacement pump.

In a second preferred embodiment, the gaseous substrate is fed to the external loop so that a gas-liquid mixture is obtained, which is subsequently sprayed into the gas phase of the bioreactor. Advantageously, in this second embodiment, the external loop may also comprise one or more static mixers to enhance gas-liquid mixing and contact under a raised pressure.

The gas mass transfer coefficient (k_{L}a) achievable with the bioreactor of the present invention was measured under physical absorption and microbial fermentation conditions to demonstrate the intensified gas mass transfer rate. The mass transfer rate was increased by 89% because of the biological intensification. In contrast to the conventional agitated bioreactors, the bioreactor of the present invention exhibits a high k_{L}a with low energy consumption. The gas mass transfer is also enhanced by a high frequency of liquid-gas contact and a small spray plume in the bioreactor. The energy consumption at a very low gassing rate is lower than that of conventional agitated bioreactors.

It has been observed that advantageously a high liquid circulation rate can be operated at a low pressure drop across the nozzles to maintain a high frequency of liquid exposure to the gas phase. This also saves energy because the low pressure drop across the nozzles dramatically reduces the energy dissipation rate. Advantageously, a high energy dissipation rate is preferably avoided in the spray bioreactor of the present invention since it cannot secure a high mass transfer rate but wastes energy.

Moreover, a large spray plume can be avoided because the gas mass transfer occurs primarily in a short distance under the nozzle tips. A small spray plume could save the cost of big spray overhead.

Additionally, the biological gas consumption by microbial cells suspended in the fermentation broth can to a great extent intensify the gas mass transfer.

Finally, the spray bioreactor of the present invention can be operated at very low gassing rates to produce PHA from CO₂, H₂ and O₂, even in the presence of diluting nitrogen and without gas circulation.

Hereinafter, the present invention is described with reference to the process of microbial CO₂ fixation in the presence of H₂ and O₂, to produce polyhydroxyalkanoates. Specifically, it has been observed that polyhydroxybutyrate (PHB) can be produced from a gas mixture of hydrogen, carbon dioxide and oxygen in the presence or absence of a large amount of nitrogen.

The process of the present invention and the related apparatus for carrying out said process, however, can also be advantageously employed in other types of microbial gas fermentations wherein sparingly soluble gaseous substrates are used.

According to a first aspect, the present invention relates to a process for producing at least one polyhydroxyalkanoate through gas fermentation which comprises the steps of:

a) providing at least one gas fermentation vessel (2) having an internal volume partially filled with a liquid fermentation broth (11) comprising:
water,
suspended gas-fermenting microorganisms capable of producing at least one polyhydroxyalkanoate,
nutrients for the gas-fermenting microorganisms;

the remaining part of the volume of the gas fermentation vessel (2) being filled with a gas phase (15);

b) continuously withdrawing an aliquot of the liquid fermentation broth (11) from the gas fermentation vessel (2);

c) supplying a gaseous substrate (14) comprising CO₂, H₂ and O₂;

d) mixing the gaseous substrate (14) with the liquid fermentation broth (11) withdrawn from the gas fermentation vessel (2) by contacting the liquid fermentation broth (11) in the form of sprayed droplets with the gaseous substrate (14) in the gas phase (15);

e) cultivating the gas-fermenting microorganisms with the gas-liquid mixture obtained in step d to form a cell mass containing at least one polyhydroxyalkanoate;

f) recovering the at least one polyhydroxyalkanoate from the cell mass.

In a preferred embodiment, the process for producing at least one polyhydroxyalkanoate through gas fermentation comprises:

a) providing at least one gas fermentation vessel (2) having an internal volume partially filled with a liquid fermentation broth (11) comprising:
water,
suspended gas-fermenting microorganisms capable of producing at least one polyhydroxyalkanoate,
nutrients for the gas-fermenting microorganisms;

the remaining part of the volume of the gas fermentation vessel (2) being filled with a gas phase (15);

b) continuously withdrawing an aliquot of the liquid fermentation broth (11) from the gas fermentation vessel (2);

c) supplying a gaseous substrate (14) comprising CO₂, H₂ and O₂ and mixing the gaseous substrate (14) with the liquid fermentation broth (11) withdrawn from the gas fermentation vessel (2);

d) spraying the so obtained gas-liquid mixture into the gas fermentation vessel (2) and cultivating the gas-fermenting microorganisms to form a cell mass containing at least one polyhydroxyalkanoate;

e) recovering the at least one polyhydroxyalkanoate from the cell mass.

In a further preferred embodiment, the process for producing at least one polyhydroxyalkanoate through gas fermentation comprises the steps of:

a) providing at least one gas fermentation vessel (2) having an internal volume partially filled with a liquid fermentation broth (11) comprising:
water,
suspended gas-fermenting microorganisms capable of producing at least one polyhydroxyalkanoate,
nutrients for the gas-fermenting microorganisms;

the remaining part of the volume of the gas fermentation vessel (2) being filled with a gas phase (15);

b) continuously withdrawing an aliquot of the liquid fermentation broth (11) from the gas fermentation vessel (2);

c) supplying a gaseous substrate (14) comprising CO₂, H₂ and O₂ into the gas phase (15);

d) spraying the liquid fermentation broth (11) withdrawn from the gas fermentation vessel (2) into the gas phase (15);

e) cultivating the gas-fermenting microorganisms with the gas-liquid mixture obtained in step d to form a cell mass containing at least one polyhydroxyalkanoate;

f) recovering the at least one polyhydroxyalkanoate from the cell mass.

Preferably, the liquid fermentation broth is withdrawn and re-introduced into the gas fermentation vessel at a circulation rate to provide a liquid retention time equal to or lower than 0.5 min, preferably equal to or lower than 0.3 min. The liquid retention time is determined by the liquid volume in the bioreactor divided by the liquid circulation rate.

Preferably, said gaseous substrate is supplied into the gas fermentation vessel with a gassing rate equal to or lower than 0.001 m/s, preferably equal to or lower than 0.0005 m/s.

Preferably, the spraying of the liquid fermentation broth or of the gas-liquid mixture formed by the gaseous substrate and the fermentation broth into the gas phase is carried out through one or more nozzles each having a pressure drop equal to or lower than 1.0-10⁵ Pa.

Preferably, the gaseous substrate is mixed with the liquid fermentation broth withdrawn from the gas fermentation vessel in at least one static mixer to form a gas-liquid mixture that is sprayed into the gas fermentation vessel.

Preferably, the gaseous substrate is a flue gas, namely a gas stream that is produced by a combustion process.

According to a further aspect, the present invention relates to a gas fermentation bioreactor (1) comprising:
a vessel (2) having an internal volume partly filled with a liquid fermentation broth (11) comprising gas-fermenting microorganisms and a fermentable medium, the remaining part of the volume of the vessel (2) being filled with a gas phase (15);
at least one effluent circulation conduit (19) communicating with the interior of the vessel (2) through which an aliquot of the liquid fermentation broth (11) is withdrawn, the conduit (19) being connected to a circulation line (6) for circulating said aliquot of the liquid fermentation broth (11) externally with respect to the vessel (2) and then re-introducing it into the gas phase (15) present in the vessel (2);
at least one spraying nozzle (12) connected to the effluent circulation line (6) for spraying the liquid fermentation broth (2) into the gas phase (15);
a feeding system (30) for feeding into the gas phase (15) a gaseous substrate (14) comprising one or more gases to cultivate the gas-fermenting microorganisms, the feeding system (30) being connected to the gas phase (15) through an inlet conduit (25) communicating with the interior of the vessel (2).

According to a further aspect, the present invention relates to a gas fermentation bioreactor (1) comprising:
a vessel (2) having an internal volume partly filled with a liquid fermentation broth (11) comprising gas-fermenting microorganisms and a fermentable medium, the remaining part of the volume of the vessel (2) being filled with a gas phase (15);
at least one effluent circulation conduit (19) communicating with the interior of the vessel (2) through which an aliquot of the liquid fermentation broth (11) is withdrawn, the conduit (19) being connected to a circulation line (6) for circulating said aliquot of the liquid fermentation broth (11) externally with respect to the vessel (2) and then re-introducing it into the gas phase (15) present in the vessel (2);
at least one spraying nozzle (12) connected to the effluent circulation line (6) for spraying the liquid fermentation broth (11) into the gas phase (15);
a feeding system (30) for feeding into the liquid fermentation broth (11) withdrawn from the vessel (2) a gaseous substrate (14) comprising one or more gases to cultivate the gas-fermenting microorganisms, the feeding system (30) being connected to the circulation line (6) so that the gas-liquid mixture thus obtained is sprayed into the gas phase (15) present in the vessel (2) by said spraying nozzle (12).

Preferably, the circulation line (6) comprises one or more static mixers (7) for mixing the liquid fermentation broth (11) withdrawn from the vessel (2) with the gaseous substrate (14) to form the gas-liquid mixture to be sprayed into the gas phase (15).

### SHORT DESCRIPTION OF THE FIGURES

FIG. 1 is a schematic structure of a spray bioreactor according to an embodiment of the present invention (SNBR);

FIG. 2 shows a schematic diagram of the spray nozzle bioreactor with two static mixers and side-line gas introduction (SNSMBR);

FIG. 3 shows the response time course of the optical DO (dissolved oxygen) probe in a step-change of dissolved oxygen concentration;

FIG. 4 shows the volumetric mass transfer coefficient and power dissipation at a superficial gas velocity of 0.00028 m·s-1 in the spray bioreactor and conventional stirred bioreactors as described in M. Nocentini, D. Fajner, G. Pasquali, F. Magelli, Ind. Eng. Chem. Res. 32 (1993) 19-26 (α=0.0083, β=0.62 and γ=0.49) and Y. Kawse, M. Moo-Yong, Chem. Eng. Res. Des. 66 (1988) 284-288 (α=0.0126, β=0.65 and γ=0.5);

FIG. 5 shows the fermentation time courses with Inlet gas I (Table 1) in the absence of nitrogen: (A) biomass concentration, residual biomass concentration and PHB content, (B) instantaneous and cumulative cell yield and dissolved oxygen concentration, and (C) specific growth rate;

FIG. 6 shows the fermentation time courses with Inlet gas III (Table 1) in the presence of nitrogen: (A) biomass concentration, residual biomass and PHB content, (B) instantaneous and cumulative cell yield and dissolved oxygen concentration (DO), and (C) specific growth rate.

FIG. 7 illustrates the schematic structure of a conventional stirred bioreactor (CSBR) for microbial fermentation.

FIG. 8 shows the time course of cell growth and increase of optical density (OD) for a fermentation carried out in the stirred bioreactor of FIG. 7.

FIG. 9 shows a schematic diagram of a conventional packed bed absorption column bioreactor (conventional gas absorber—CGA).

FIG. 10 shows the time course of cell growth and increase of optical density (OD) for a fermentation carried out in the conventional gas absorber of FIG. 9.

FIG. 11 shows the time course of cell growth and increase of optical density (OD) for a fermentation carried out in the spray nozzle bioreactor SNBR of FIG. 1;

FIG. 12 shows the time course of cell growth and increase of optical density (OD) for a fermentation carried out in the spray nozzle bioreactor according to FIG. 2.

### DETAILED DESCRIPTION

Across a gas-liquid interphase, mass transfer occurs under a concentration driving force. According to the two film resistance theory, the mass transfer resistance of a sparingly soluble gas such as O₂, H₂ and CO₂ is mainly located in the liquid film and the mass flux (Ni) is governed by Eq. 1:

$${N}_{i} = {k}_{i , L} ( {C}_{i}^{*} - {C}_{i , L} ) = {k}_{i , L} ( \frac{{P}_{i}}{{H}_{i}} - {C}_{i , L} ) \qquad ( Eq . 1 )$$

where k_{i,L} is the liquid phase mass transfer coefficient of gas “i”, Pᵢ the partial pressure in the gas phase, Hᵢ the Henry's constant, C_{i,L}* is the equilibrium concentration (kmole m⁻³) and C_{i,L} the concentration in the liquid phase. The Whitman's film model also indicates that the mass transfer coefficient (k_{i,L}) is determined by the molecular diffusivity (D_{i,L}) of gas “i” in water and the liquid side mass transfer resistance (R_{L}) as shown in Eq. 2

$${k}_{i , L} = \frac{{D}_{i , L}}{{R}_{L}} . \qquad ( Eq . 2 )$$

In this work, only the volumetric mass transfer coefficient of O₂ (k_{L}a) was measured by using a reliable probe, but k_{L}a of oxygen can be used to find those of H₂ and CO₂ from their molecular diffusivities in water (Eq. 3)

$$\frac{{k}_{i , L} a}{{k}_{L} a} = \frac{{k}_{i , L}}{{k}_{L}} = \frac{{D}_{i , L}}{{D}_{L}} . \qquad ( Eq . 3 )$$

The volumetric oxygen utilization rate (OUR) in a spray bioreactor is determined by the oxygen flux (N) expressed by Eq. 1, the total effective interfacial area (Aₑ) and the liquid volume (V_{L}) (Eq. 4)

$$OUR = N ( \frac{{A}_{e}}{{V}_{L}} ) = {k}_{L} a ( \frac{{P}_{o}}{{H}_{o}} - {C}_{L} ) = {k}_{L} a ( {C}_{L}^{*} - {C}_{L} ) \qquad ( Eq . 4 )$$

where “a” is the effective interfacial area per liquid volume (Aₑ/V_{L}), Pₒ and Hₒ are the partial pressure and Henry's constant of oxygen, C*_{L} the equilibrium concentration of oxygen under Pₒ, and C_{L} the dissolved oxygen concentration in liquid phase.

Like chemical reactions, the biological gas consumption by microbial cells in liquid solution may also reduce the mass transfer resistance (R_{L}), and hence enhance the mass transfer coefficient (k_{L}a). An enhancement factor (E) is defined as (Eq. 5)

$$E = \frac{{( {k}_{L} a )}_{Bio}}{{( {k}_{L} a )}_{Phy}} \qquad ( Eq . 5 )$$

where the subscripts Bio and Phy indicates the k_{L}a values under the conditions of biological gas utilization and physical absorption in water, respectively.

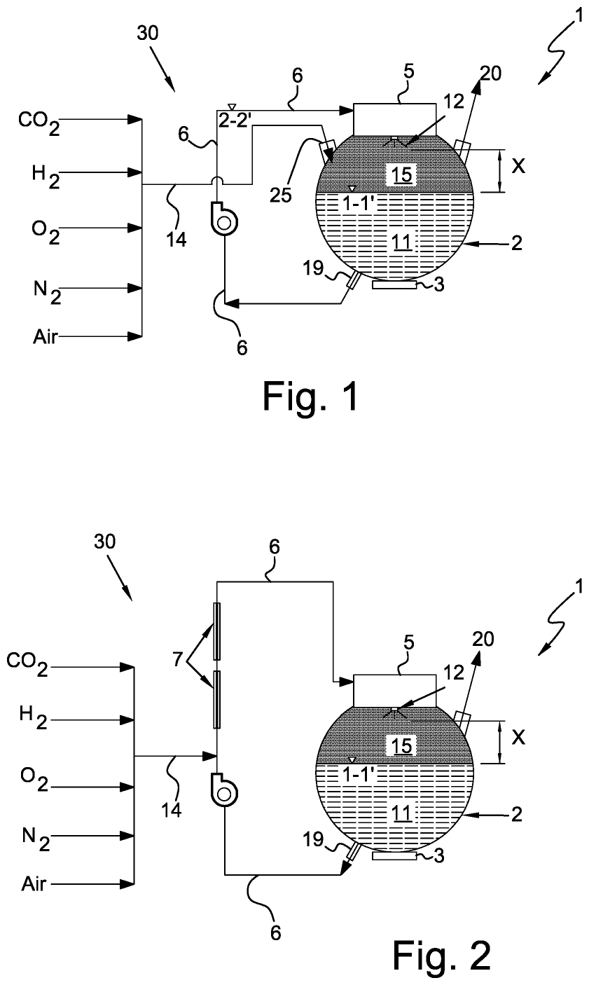

FIGS. 1 and 2 are schematic representations of a nozzle spray bioreactor 1 according to two embodiments of the present invention. A round glass vessel 2 (φ15 cm, 3 L) at the bottom held an aqueous solution (fermentation broth) 11 (0.5-1.5 L) which was agitated and heated on a magnetic stir and heater 3. The gaseous substrate 14 can be introduced into the bioreactor in two ways. With a SNBR configuration according to FIG. 1, the gaseous substrate 14 is supplied into the vessel 2 through the conduit 25. In a SNSMBR configuration according to FIG. 2, the gaseous substrate 14 is introduced into the external loop 6.

In a gas fermentation demonstration, the solution pH was controlled with a base solution (2 M ammonia or 2 M NaOH). A diaphragm pump 4 (maximum flowrate 4.9 L·min⁻¹) connected to an effluent discharge conduit 19 delivered the liquid fermentation broth to the nozzles 12 installed on the top of a plexiglass cylinder 5 ((20 cm×23 cm, 6 L). The liquid was sprayed into full cone plume (90°) into the gas phase 15 present in the vessel 2 and returned to the liquid pool 11. A gas stream 14 was introduced into the liquid circulation line 6 by means of a feeding system 30 and was sprayed with the liquid through the nozzle(s) 12 into the gas phase 15. In the embodiment of FIG. 2, the gas stream 14 was introduced into the liquid circulation line 6, by means of a feeding system 30, before two static mixers 7 and was sprayed with the liquid through the nozzle(s) 12 into the gas phase 15 present in the vessel 2. In both these embodiments, the gas flowrate and composition were controlled by the flow rates of individual gases. Air and N₂ were controlled with needle valves and flowmeters (Cole-Parmer, Vernon Hills, Ill.). CO₂, H₂ and O₂ were controlled with three mass flow meters (Alicat Scientific, Tucson, Ariz.). The bioreactor was operated under one atmosphere and the flowrate of the exiting gas 20 was measured with a soap film meter and released into a fume hood via a vent tubing. In a blank control without microbial gas consumption, the inlet gas and outlet gas had an equal molar flowrate (<+±5%).

The effect of the spray plume on mass transfer was also examined when the nozzle(s) were installed directly on the top of glass vessel. The distance “X” from the nozzle tip to the liquid surface was reduced to about 10 cm, and the volume of spray plume was reduced by 80%.

Physical Absorption of Oxygen in Water

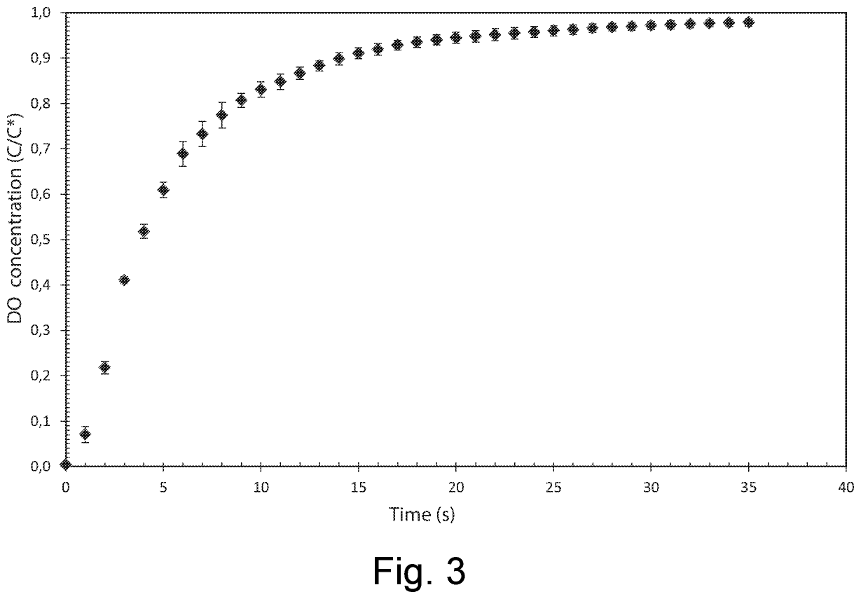

The k_{L}a of physical absorption of oxygen in water or mineral solution (i.e. the fermentable medium comprising water and the nutrients for cultivating the microorganisms) was measured in a dynamic method. The liquid solution (1.5 L) was stripped with N₂ till the dissolved oxygen concentration dropped to zero. Air was then introduced at 0.3 L min⁻¹ (25° C., 1 atm). The concentration of dissolved oxygen was monitored by using an optical DO probe (ProODO, YSI, Yellow Springs, Ohio). The k_{L}a was calculated with Eq. 6

$$ln ( \frac{{C}_{L}^{*} - {C}_{L , 0}}{{C}_{L}^{*} - {C}_{L}} ) = ( {k}_{L} a ) t \qquad ( Eq . 6 )$$

where C_{L}* is the saturated oxygen concentration under air, C_{L} the dissolved oxygen concentration at time t, and C_{L,0} the initial oxygen concentration when the measurement was recorded. The response time of the probe is defined as the time taken for the probe to reach 63.2% of the final value when exposed to a step change in concentration. When the response time is less than or equal to (k_{L}a)⁻¹, the measurement could be reliable. FIG. 3 is the response time curve of the DO probe when it was exposed to a step change in dissolved oxygen concentration. The response time of the probe in the experimental conditions was about 6 s and hence the maximum k_{L}a which could be measured in the dynamic method was 0.17 s⁻¹ or 600 hr⁻¹.

Microbial Enhancement of Oxygen Mass Transfer

The oxygen mass transfer rate in microbial gas fermentation was determined by measuring the oxygen utilization rate (OUR) under steady (or quasi steady) operation conditions. As fermentation broth, it was used a laboratory strain of C. necator grown in a mineral solution containing (per liter): 2.4 g KH₂PO₄, 2.5 g Na₂HPO₄, 1.2 g (NH₄)₂SO₄, 0.5 g MgSO₄.7H₂O, 1.0 g NaCl, 0.5 g NaHCO₃, 0.1 g ferric ammonium citrate and 1 mL of trace element solution. The medium solution was inoculated to an initial optical density (OD) of 0.91 with 100 mL bottle culture prepared in the same medium solution. The bottle (1 L) was flushed with a gas mixture of H₂ (70%), O₂ (20%) and CO₂ (10%) every 24 hours and shaken at 200 rpm and 30° C. in a rotary incubator.

The medium solution (0.5 L) in the spray bioreactor was stirred at 150 rpm and its temperature and pH were maintained at 35° C. and 6.8, respectively. The liquid was circulated at 1.2 L/min with a diaphragm pump (NF300, KNF Neuberger Inc, USA) and sprayed through a nozzle (1.65 mm orifice diameter, 90° full cone). The retention time in the liquid pool was controlled at 0.4 min. The inlet gas was set at 100 sccm (standard cubic centimeter per min at 0° C. and 1 atm), including H₂ (70 sccm), O₂ (20 sccm) and CO₂ (10 sccm). The gas molar flowrate (F) and oxygen molar fraction (y) were measured to find the volumetric oxygen utilization rate (OUR) (Eq. 7a)

$$OUR = \frac{{( Fy )}_{in} - {( Fy )}_{out}}{{V}_{L}} . \qquad ( Eq . 7 a )$$

Because of the good mixing of both liquid and gas in the bioreactor, the liquid solution was homogenous and the partial pressure of oxygen (P_{O}) in the gas phase was equal to that of outlet gas stream. When the dissolved oxygen concentration (C_{L}) in Eq. 4 dropped to zero, the oxygen mass transfer became the rate-limiting step of oxygen utilization. The maximum OUR therefore reflects the k_{L}a of the spray bioreactor (Eq. 7b)

$${OUR}_{max} = {k}_{L} a ( \frac{{P}_{o}}{{H}_{o}} ) . \qquad ( Eq . 7 b )$$

Microbial CO₂ Fixation and PHB Biosynthesis

PHB biosynthesis from CO₂ was conducted in the spray bioreactor with reduced spray plume by installing the nozzles (10 mm² open area) on the bottom glass vessel. The liquid medium (1.5 L) was maintained at 35° C. and circulated at 4.7 L min⁻¹ under a pressure drop of 48 kPa across the nozzles. The medium pH was maintained at 6.8 by using an ammonia solution (2 M) at the beginning to provide nitrogen nutrient and then a NaOH solution (2 M) to promote PHB formation. The inlet gas composition and flow rate in each fermentation were maintained at constant levels as shown in Table 1. N₂ gas (40-60% vol/vol) was introduced into the bioreactor to examine its dilution effect on gas fermentation.

TABLE 1
Inlet gas composition and flow rate in
gas fermentations for PHB production

| | Flow rate | H₂ | O₂ | CO₂ | N₂ |
|---|---|---|---|---|---|
| Inlet gas | (sccm)* | (mole %) | (mole %) | (mole %) | (mole %) |
| I | 140 | 60.0 | 20.0 | 20.0 | 0 |
| II | 70 | 60.0 | 20.0 | 20.0 | 0 |
| III | 350 | 40.0 | 10.8 | 9.0 | 40.2 |
| IV | 350 | 10.0 | 16.2 | 13.5 | 60.3 |

*Standard cubic centimeter per min (sccm) at 0° C. and 100 kPa

Chemical Analysis

The gas composition was determined by using a gas chromatograph (Model 450, Bruker, Fremont, Calif.). The GC was equipped with a thermal conductivity detector (TCD) and a Carboxen PLOT 1006 column (0.15 mm×30 m, Sigma-Aldrich, St Louis, Mo.). The temperature cycle was started at 35° C. for two mins and then raised to 100° C. in 3 mins. The peaks were integrated using software Galaxie and calibrated with gas standards. The standard gas samples of different molar composition were prepared by using a gastight syringe from pure gas and nitrogen as the background gas.

The microbial growth was monitored by measuring the optical density (OD) of the medium solution at 620 nm with a UV/Vis spectrophotometer (DU530, Beckman-Coulter, Fullerton, Calif.). The medium solution was centrifuged at 5,000 g for 5 min and the wet pellets were freeze-dried to determine the dry cell mass concentration. The PHB content of the cell mass was determined by using the GC with a flame ionization detector (FID) on a sample prepared as follows: about 50 mg of PHB-containing dry cell mass was added in 2 mL methanol solution (H₂SO₄ 3% v/v, benzoic acid 10 g L⁻¹ as the internal standard) and 2 mL chloroform, and maintained at 100° C. for 4 hours. The intracellular polyester was converted into 3-hydroxybutyrate methyl ester and released into the reaction solution. After the mixture was cooled to room temperature, 1 mL distilled water was added and the solution was vortexed for 1 min. The mixture was allowed to separate into an aqueous and an organic phase. The organic solution was filtered through a PTFE membrane (0.45 μm) and analyzed with GC. A pure PHB (Sigma-Aldrich, St Louis, Mo.) was treated in the same procedure for calibration.

Physical Absorption of Oxygen in Water

Table 2 gives the k_{L}a of physical absorption of oxygen in water observed under different operation conditions.

TABLE 2
Physical absorption and mass transfer of oxygen in water a

| | Orifice | Open | Circulation | Pressure | Droplets | | Power |
|---|---|---|---|---|---|---|---|
| | diameter | area | rate | drop^{c} | velocity^{d} | k_{L}a | dissipation |
| Nozzles^{b} | (mm) | (mm²) | (L · min − 1) | (× 10⁵ Pa) | (m · s − 1) | (s − 1) | (kw · m − 3) |
| 1 | 1.44 | 1.63 | 2.4 | 2.57 | 22.7 | 0.031 | 6.9 |
| 1 | 1.65 | 2.14 | 2.8 | 2.38 | 21.9 | 0.033 | 7.3 |
| 1^{e} | 2.06 | 3.33 | 3.5 | 1.92 | 19.6 | 0.044 | 7.5 |
| 2 | 2.06 | 6.66 | 4.6 | 0.61 | 11.0 | 0.062 | 3.3 |
| 3 | 2.06 | 9.99 | 4.7 | 0.48 | 9.8 | 0.064 | 2.7 |
| 3^{f} | 2.06 | 9.99 | 4.7 | 0.48 | 9.8 | 0.075 | 2.7 |

a. Aeration 0.2 vvm (gas volume per liquid volume per min): 1.5 L water with 0.3 L · min −1 air
^{b}90° full cone nozzles
^{c}Pressure drop (ΔP) across the nozzle(s)
${}^{d} Initial velocity of droplets from nozzles ( {U}_{0} = Cp \sqrt{\frac{2 Δ P}{{ρ}_{L}}} , Cp = 0.95 )$ ^{e}The same liquid retention time (0.4 min) as in the experiment of biological enhancement
^{f}The same nozzles and operations with reduced spray plume.

The superficial gas velocity was 0.00028 m s⁻¹, or 0.2 vvm (gas volume per liquid volume per min) in terms of liquid gassing rate. When one nozzle was used, the k_{L}a value (0.03-0.04 s⁻¹) was in the range of conventional aerated bioreactors.

The k_{L}a was almost doubled (0.064 s⁻¹) when multiple nozzles were used to give a larger open area. Interestingly, this increase in k_{L}a was observed with decrease in the pressure drop across the nozzle(s) and the velocity of liquid droplets, a phenomenon contrast to conventional knowledge. According to the mechanical energy balance around the nozzle(s), a high pressure drop would generate a high velocity and flow turbulence of liquid droplets in the gas phase. The high turbulence would also generate smaller droplets to give high gas-liquid interfacial area. All these factors should intensify the gas mass transfer rate, but instead a low value of k_{L}a was observed. Without being bound to any theory, it is believed that this conventional analysis may be true for individual liquid droplets, but not applicable to the liquid pool or whole bioreactor. It was observed that the liquid circulation rate of the pump declined with a smaller nozzle open area, which reduced the frequency of liquid exposure to the gas phase. At a high circulation rate (4.7 L·min⁻¹), the pressure drop and liquid velocity were the lowest, but the retention time in the liquid pool (1.5 L) was the shortest (0.32 min), i.e. the frequency of liquid-gas contact was the highest. To intensify the gas mass transfer in spray bioreactors, a high liquid circulation rate associated with a low pressure drop is therefore preferred rather than a low circulation rate associated with a high pressure drop. This is also true for energy saving as shown latter.

When multiple nozzles were used for spraying, the plume was larger and contained more liquid droplets than a single nozzle did. This raised a question whether or not a large spray plume should be provided to intensify the gas mass transfer. This was examined when the top cylinder was removed and the nozzles were installed directly on the top of glass vessel. The spray plume was reduced dramatically from 7.5 L to 1.5 L. Under the same operation conditions, however, the k_{L}a reached 0.075 s⁻¹ or increased by 17% as shown in Table 2. In other words, the small plume did not reduce, but intensified the gas mass transfer. This might be attributed to a fact that gas-liquid mass transfer occurs primarily under the tip of nozzles. A long retention time of small liquid droplets in the gas phase did not make a substantial contribution to mass transfer. Instead, the impact of the liquid droplets at free liquid surface could to some extent intensify the gas mass transfer.

Microbial Enhancement of Oxygen Mass Transfer

Table 3 gives the measurements of oxygen utilization rate (OUR) and k_{L}a at different time points in a gas fermentation.

TABLE 3
Intensified oxygen mass transfer in
spray bioreactor with one nozzle ^{a}

| | | Dis- | Outlet | Outlet | OUR | | |
|---|---|---|---|---|---|---|---|
| | | solved | gas ^{c} | O₂ | (mmole · | | |
| Time | | O₂ (mg · | (mL · | (mole | L⁻¹ · | k_{L}a | E ^{d} |
| (hr) | OD ^{b} | L⁻¹) | min⁻¹) | %) | min⁻¹) | (s⁻¹) | (—) |
| 0 | 1 | 6.43 | 104 | 20.64 | 0 | 0 | — |
| 16 | 2.1 | 2.25 | 75 | 21.34 | 0.45 | 0.042 | 0.95 |
| 24 | 6 | 0.25 | 64 | 21.58 | 0.64 | 0.055 | 1.25 |
| 32 | 15.1 | 0 | 51 | 17.83 | 0.96 | 0.083 | 1.89 |
| 40 | 25.1 | 0 | 60 | 16.62 | 0.86 | 0.074 | 1.68 |
| 48 | 23.6 | 0 | 62 | 19.84 | 0.75 | 0.073 | 1.66 |

^{a} Nozzle orifice diameter ϕ1.65 mm, open area 2.14 mm², 90° full cone
^{b} Optical density of culture medium at 600 nm (1 OD = 0.4 g dry cell mass/L)
^{c} The outlet volumetric gas flow rate (25° C., 1 atm)
^{d} Comparison with physical absorption of oxygen in water (k_{L}a = 0.044 s⁻¹) in Table 2.

To maintain a good mixing and frequent exposure of liquid to the gas phase, the medium solution (0.5 L) was stirred at 100 rpm and circulated at 1.2 L/min to give a liquid retention time of 0.4 min in the pool. The pressure drop across the nozzle (φ1.65 mm) was 90 kPa and did not change with increase of cell density. Under the operation conditions, the energy dissipation in the liquid solution from the pump was 3.6 kw m⁻³. The optical density (OD) increased from 1 to 25, or the biomass concentration from 0.4 g/L to 10 g/L. With sufficient nitrogen nutrient, the microbial cells formed little PHA (<2%) and the reduced CO₂ was primarily used for cell growth. The elemental composition of the cell mass, which was determined as described in S. Kang and J. Yu, Biomass Bioenergy, 74 (2015) 92-95, was: 45.1% C, 6.3% H, 12.8% N, 27.8% O and 8.0% ash, from which the organic formula is CH_{1.69}O_{0.46}N_{0.25}. Because of microbial gas consumption, the outlet gas flow rate dropped from 104 mL/min at the beginning to 50-60 mL/min. The bioreactor overhead (about 9 L) was flushed for 8 hours by the existing gas before next measurement. Each gas flowrate and composition were the averages of three measurements taken within 30 minutes. Because of the relatively slow biological activity, quasi steady state of gas composition was assumed.

From the oxygen utilization rate, k_{L}a was calculated with Eq. 7. The value of k_{L}a was low at low ODs because of low oxygen utilization rate, and then reached the highest level when the dissolved O₂ concentration dropped to zero, a clear indicator of oxygen mass transfer limitation. In other word, the highest k_{L}a (0.083 s⁻¹) reflected the maximum oxygen mass transfer coefficient of the spray bioreactor under the operation conditions. The k_{L}a declined with further increase in cell density. This is attributable to a low OUR because of the reduced microbial activity after the obligate aerobic strain had been exposed to oxygen depletion for a long time. Compared with the k_{L}a (0.044 s⁻¹) of physical absorption in water at the same liquid retention time (0.4 min) (Table 2), the enhancement factor of oxygen mass transfer by microbial activity was calculated and listed in Table 3. The gas mass transfer was intensified to a great extent by the biological consumption of oxygen (and other two gases, too) by the microbial cells.

Energy Consumption and Comparison The spray bioreactor has a simple structure. The pump in liquid circulation line provides the mechanical energy for both liquid spray and mixing. A mechanical energy balance between two points (1-1′ and 2-2′) in FIG. 1 shows that the energy input (Wₛ) from the pump minus friction loss is equal to the increase of kinetic energy, potential energy and pressure energy of the fluid (Eq. 8a)

$${W}_{S} - Σ F = \frac{( {U}_{2}^{2} - {U}_{1}^{2} )}{2} + g ( {Z}_{2} - {Z}_{1} ) + \frac{( {P}_{2} - {P}_{1} )}{{ρ}_{L}} ( J \text{/} kg ) . \qquad ( Eq . 8 a )$$

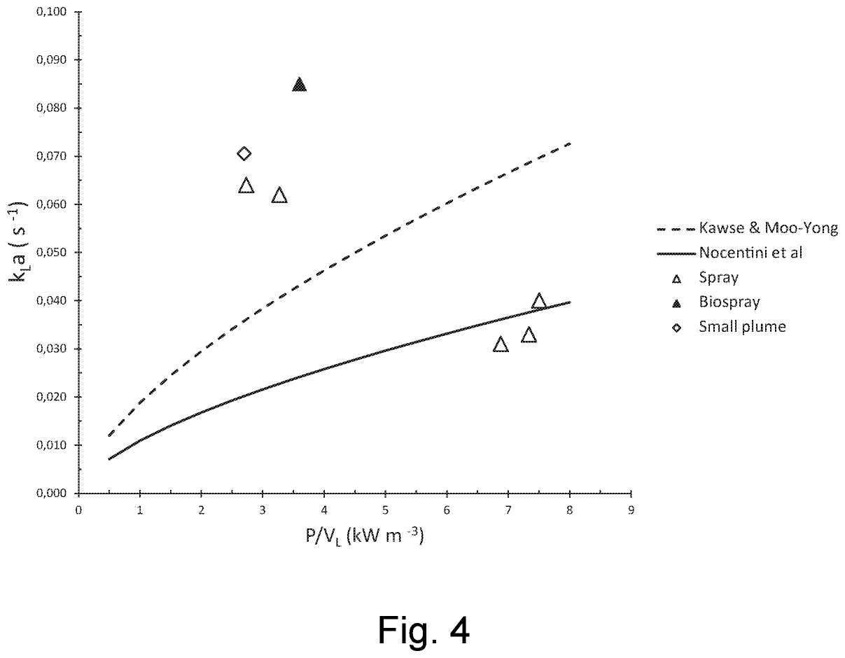

With the negligible friction loss, U₁ and height difference between two points, it is simplified as Eq. 8b

$${W}_{S} = \frac{{U}_{2}^{2}}{2} + \frac{Δ P}{{ρ}_{L}} , \qquad ( Eq . 8 b )$$

where U₂ is the liquid velocity in the tubing (inner diameter 6 mm) and ΔP the pressure drop across the nozzle. Under the experimental conditions (Table 2), the dynamic energy accounted only for a small portion (<8%) of the pump input. The energy was primarily used to raise the pressure of liquid for spray. The power consumption per liquid volume was calculated from the liquid circulation rate and pressure drop with Eq. 8b. As shown in Table 2, a high liquid circulation rate under low pressure drop has a much lower energy dissipation (2.7 kw·m⁻³) than a low circulation rate under a high pressure drop (7.3 kw·m⁻³). In the latter case, the energy was dissipated as surface energy and heat in formation of tiny liquid droplets (<50 um) even though the high interfacial area does not contribute very much to the gas mass transfer rate as mentioned above.
In conventional agitated bioreactors, the oxygen mass transfer coefficient is dependent on superficial gas velocity as well as the power dissipation in liquid solution. A widely used correlation for stirred vessels has a general form (Eq. 9)

$${k}_{L} a = {α ( \frac{P}{{V}_{L}} )}^{β} {U}_{S}^{γ} , \qquad ( Eq . 9 )$$

where k_{L}a is the oxygen mass-transfer coefficient in units of s⁻¹. P is the power dissipated by the stirrer in W, and V_{L} is the liquid volume in m³. Uₛ is the superficial gas velocity in m s⁻¹ and defined as the volumetric gas flow rate divided by the cross-sectional area of the bioreactor. Numerous correlations have been obtained from experimental data and/or derived from theory. Two of them are plotted in FIG. 4 for comparison: one from experimental correlation (M. Nocentini et al., Ind. Eng. Chem. Res. 32 (1993) 19-26) and another from Kolmogorov's theory which is not restricted to special configuration and experimental conditions (Y. Kawse, M. Moo-Yong, Chem. Eng. Res. Des. 66 (1988) 284-288). Most of the experimental correlations gave quite consistent predictions on k_{L}a while the theory based correlation gives a higher prediction (M. Xie, J. Xia, Z. Zhou, J. Chu, Y. Zhuang, S. Zhang, Ind. Eng. Chem. Res. 53 (2014) 5941-5953). They are compared with the k_{L}a of spray bioreactor at the same energy dissipation rate in FIG. 4. It should be pointed out that the empirical correlations are obtained from special experiments in which the minimum superficial gas velocity is 0.001 m s⁻¹ or above. When the correlations are used at a very low gas velocity in the present description (0.00028 m s⁻¹ in Tables 2 and 3), they are only for reference purpose.

FIG. 4 revealed some interesting information. First, when the spray bioreactor is operated at a high pressure drop across the nozzles, the high energy dissipation cannot secure a high mass transfer coefficient. With high energy dissipation, the spray bioreactor exhibits the similar k_{L}a of conventional bioreactor at very low gassing rates. Second, with low energy dissipation, spray bioreactors can provide higher mass transfer rates than conventional bioreactors. At a very low gassing rate, mechanical energy is more efficiently used to break down liquid into droplets than to break down gas bubbles. Spray bioreactor can therefore use the same amount of energy to provide a higher k_{L}a. Third, a reduced spray plume promotes mass transfer in spray bioreactors. Finally, biological gas consumption can enhance the gas mass transfer in the medium solution.

PHB Formation from CO₂ in the Absence of Nitrogen

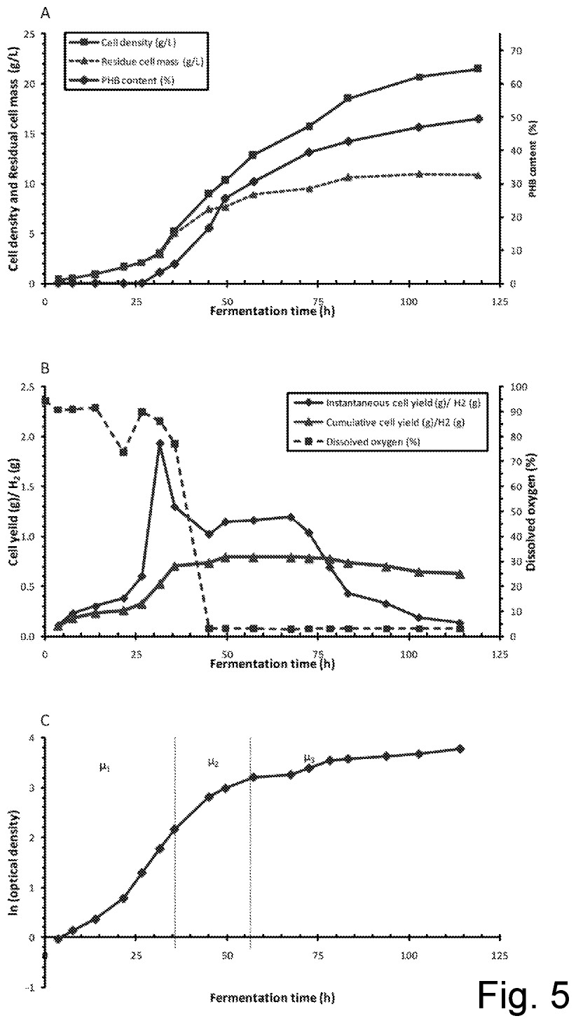

FIG. 5 is the fermentation time courses of Inlet gas I (Table 1). At a constant flow rate (140 sccm) of H₂ (60%), O₂ (20%) and CO₂ (20%), the microbial cell density increased continuously with time and reached a maximum level of 21.5 g/L. A large portion of the reduced carbon was stored in PHB and the final PHB content reached 50 wt %. Interestingly, the residual biomass, the mass difference between the cell mass and PHB, increased at the beginning with sufficient nitrogen nutrient and approached a plateau when a nitrogen limitation was applied. This pattern was similar to those of heterotrophic cultures on organic carbon source as described for example in N. Tanadchangsaeng, J. Yu, Biotechnol. Bioeng. 109 (2012) 2808-2818. The dissolved oxygen (DO) concentration in the liquid solution was monitored as percentage of air saturation and kept at a quite high level (>70%) till the cell density reached 5 g/L. During that period of time, the oxygen mass transfer rate was high enough to support the high microbial activity. When the biomass concentration increased to 9 g/L or above, the DO dropped quickly to zero and the obligate aerobic strain was exposed to the condition of oxygen depletion thereafter. The availability of gas substrates affected the microbial growth and physiology. The microbial growth could be roughly divided into three stages according to the specific growth rates (μ). In FIG. 5, the specific growth rate is presented as the curve slope of natural logarithm of optical density versus time. After an initial lag phase, the specific growth rate was 0.1 h⁻¹ (μ₁) and then declined because of oxygen limitation at high cell densities. It was reduced to 0.06 h⁻¹ (μ₂) and then further to 0.01 h⁻¹ (μ₃) as fermentation continued for 115 h.

FIG. 5 also gives the change of instantaneous and accumulative hydrogen yield with time. Hydrogen was the only source of energy and reducing agent for microbial CO₂ fixation. The hydrogen yield is defined as the amount of biomass formed per hydrogen fed (g·g⁻¹). The “quasi” instantaneous yield, measured from the difference of two time points, indicates an average biological efficiency of CO₂ fixation in this short period of time. The accumulative hydrogen yield reflects the overall efficiency from the beginning of a fermentation to a time point. The instantaneous hydrogen yield was the highest (about 2 g·g⁻¹) corresponding to the maximum specific growth rate (0.1 s⁻¹), and then kept at a moderate level (1.2 g·g⁻¹) for a quite long time during which most of cell mass was formed. It dropped to zero because little cell mass was formed. As an important criteria of the process economy, the accumulative hydrogen yield of whole batch fermentation was 0.6 g·g⁻¹.

With Inlet gas II of the same gas composition, the gas flowrate was reduced to 70 sccm and similar time courses were observed. However, the maximum biomass concentration was reduced to 12.4 g/L and PHB content reduced to 17.5 wt %. The maximum instantaneous hydrogen yield was 1.8 g·g⁻¹, indicating the similar mass transfer rate and CO₂ fixation efficiency. The accumulative hydrogen yield was much higher (0.95 g·g⁻¹) because of lower hydrogen wastage at the very low gassing rate (0.05 vvm). With Inlet gas B, the microbial cells might not have sufficient carbon and energy source for growth.

The Effect of Nitrogen on PHB Formation from CO₂

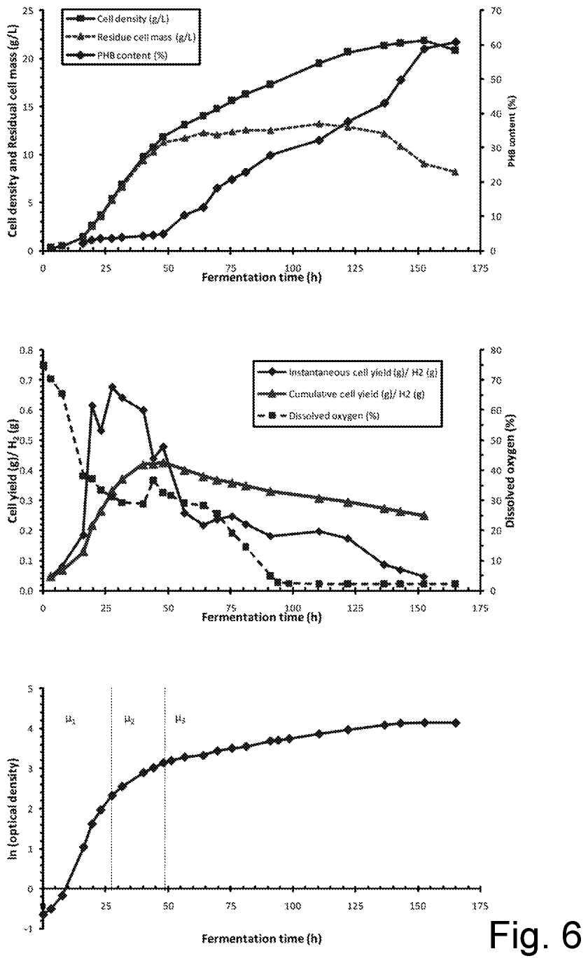

FIG. 6 is the fermentation time course of Inlet gas III (Table 1) in the presence of a large amount of N₂ gas (40 mole %). A high biomass density of 21.9 g/L was also achieved and the PHB content was as high as 61 wt %. The high PHB content could be attributed to the decline of residual biomass after a long time at a plateau. Interestingly, the dissolved oxygen was kept at a high level (15-30%) for up to 75 hours during which a large amount of cell mass (16 g/L) was formed. It dropped to zero when cell density reached 17 g/L. Obviously, the obligate aerobic strain had sufficient oxygen to maintain good metabolic activity for a quite long time. The maximum specific growth rate (μₘₐₓ) was 0.12 h⁻¹ (μ₁) for a period of 20 h after an initial lag phase of 8 h. The growth rate declined to 0.04 h⁻¹(μ₂) as the fermentation progressed for additional 20 hours or so. Finally, the specific growth rate was kept at 0.01 h⁻¹ (μ₃) till the end of the fermentation. The hydrogen yields, however, were lower in the presence of N₂ which diluted the gas composition. The highest instantaneous hydrogen yield was 0.7 g·g⁻¹ and the accumulative yield was 0.3 g·g⁻¹. This experiment shows that it is possible to use flue gas directly as the CO₂ source, which saves the cost of CO₂ capture.

When a very small amount of hydrogen (10 mole %) was provided in Inlet gas IV, little cell growth was observed. The maximum optical density (OD) after 32 hours was only 1.68 (cell density≈0.81 g/L). The cell density was maintained at this level at the same gassing rate throughout the operation. It seems that the low H₂ composition was just enough for cell maintenance without sufficient H₂ available for CO₂ fixation and cell growth. Table 4 compares the gas fermentation of different inlet gases. The results of Inlet gas C and D clearly indicate the dilution effect of N₂ (40-60 mol %).

TABLE 4
The effects of inlet gas on microbial
CO₂ fixation and PHB synthesis

| Inlet gas ^{a} | I | II | III | IV |
|---|---|---|---|---|
| Maximum specific growth rate (hr⁻¹) | 0.10 | 0.07 | 0.12 | n/a |
| Maximum hydrogen yield (g · g⁻¹)^{b} | 2.0 | 1.8 | 0.7 | n/a |
| Cumulative hydrogen yield (g · g⁻¹)^{b} | 0.6 | 0.95 | 0.24 | n/a |
| Maximum cell density (g · L⁻¹) | 21.5 | 12.4 | 21.9 | 0.8 |
| PHB content (wt %)^{c} | 50 | 17.5 | 61 | n/a |

^{a} Gas composition and flowrates can be found in Table 1
^{b}Grams of dry cell mass formed per gram of hydrogen fed
^{c}Weight percentage of PHB in dry cell mass

Herewith following, the performance of the bioreactor according to the present invention for cell growth and PHB formation from a gas substrate is compared with that of two conventional bioreactor and gas absorber.

Performance of Conventional Bioreactors for Microbial Gas Culture

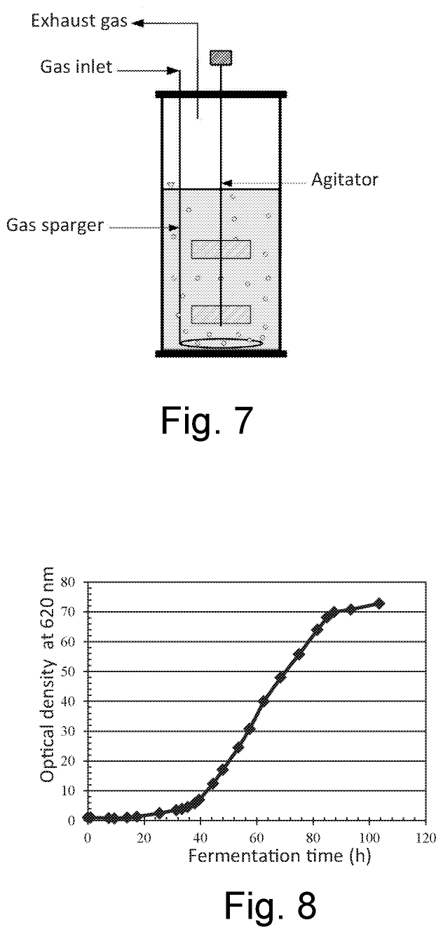

A conventional bench-top bioreactor (3 liters total volume, BioFlo 110, New Burnswick, Enfield, Conn.) was used to cultivate C. necator on a gas mixture of 70 mol % H₂, 20 mol % O₂ and 10 mol % CO₂. FIG. 7 illustrates the schematic structure of the reactor, a very popular bioreactor for microbial fermentation. The gas mass transfer from the bubbles to the suspended microbial cells is provided via agitation of the medium solution by a mechanical agitator. A mineral solution (1.5 L) consisting of potassium phosphate 2.3 g/L, sodium monohydrogen phosphate 4.37 g/L, ammonium chloride 1 g/L, magnesium sulfate 0.5 g/L, sodium bicarbonate 0.5 g/L, ferric ammonium citrate (FAC) 0.05 g/L, calcium chloride dehydrate 0.01 g/L and trace mineral 1 mL of previously prepared solution was used as the liquid culture medium. The agitation rate was maintained at 900 rpm in order to provide the maximum mass transfer. Gas sparger was used to disperse the gas stream into the solution. Once the culture medium in the bioreactor reached stable condition (pH and temperature) the bottle culture was inoculated. The pH and temperature were maintained at 6.9 and 30° C., respectively. Operation conditions such as DO, pH, temperature and agitation speed were monitored. Exhaust gas was monitored using a gas analyzer (TANDEM Pro, Magellan Instruments Ltd, Norfolk, UK). Liquid samples were collected and analyzed for optical density (OD at 620 nm), cell density, and PHB content.

The volumetric mass transfer coefficient (k_{L}a) of O₂ from an air stream (21 mol % O₂) was measured in the same solution above and used as a bench marker for comparison of gas mass transfer in different bioreactors. Table 5 shows the results of this conventional bioreactor. In fermentation industry, the gas flow rate is often expressed as ‘vvm’, or gas volume per liquid volume per min. The maximum k_{L}a of 234 h⁻¹ was obtained at 900 rpm with an air flow rate of 4.5 L/min (3.0 vvm). Reducing the air flow rate caused a significant reduction in k_{L}a. For example, at the air flow rate of 0.75 L/min (0.5 vvm) and 900 rpm agitation speed, the k_{L}a was only 144 h⁻¹, or a reduction of 38%.

TABLE 5
Volumetric mass transfer coefficients (k_{L}a) of
oxygen in the conventional bioreactor of FIG. 7

| Agitation | Volumetric mass transfer coefficient (k_{L}a)(h⁻¹) | | | |
|---|---|---|---|---|
| speed (rpm) | 0.5 vvm^{a} | 1.0 vvm | 2.0 vvm | 3.0 vvm |
| 200 | 14.8 | 18.4 | 21.6 | 27.0 |
| 500 | 74.2 | 75.2 | 86.2 | 91.4 |
| 900 | 144.4 | 170.6 | 215.6 | 234.4 |

^{a}vvm = volume of gas at standard conditions per unit liquid volume per minute, liquid volume of the reactor is 1.5 L.

FIG. 8 shows the variation of optical density with fermentation time. The maximum OD of 72.8 was observed after 103 hours of fermentation. The starting dissolved oxygen (DO) was around 75% and gradually it dropped to 0% after 45 h of operation, indicating the cell growth was limited by gas mass transfer rate. The maximum specific growth rate (μₘₐₓ) of 0.08 h⁻¹ was observed from 20 to 60 h of the fermentation. Then the specific growth rate reduced to 0.03 h⁻¹ till it reached a plateau. At the end of the experiment, the maximum cell density and the PHB content were recorded as 24.16 g/L and 59%, respectively.

Conventional Gas Absorber for Microbial Gas Culture

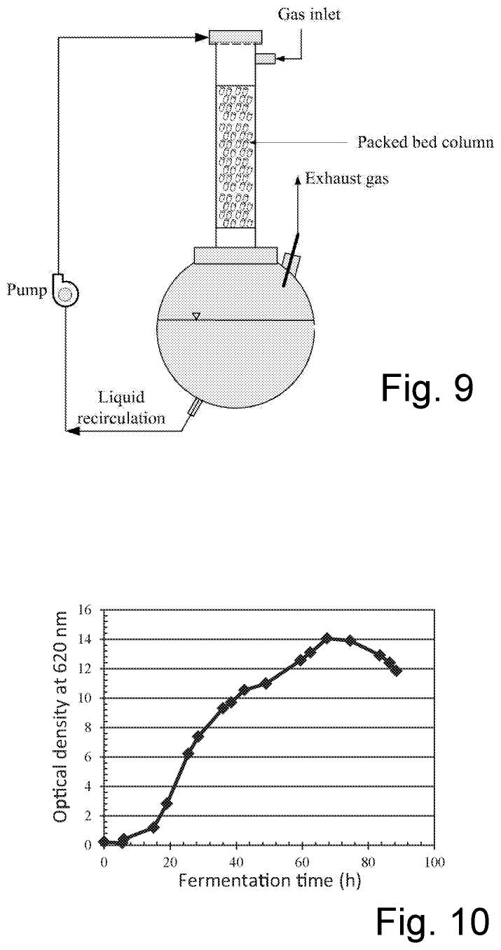

A conventional gas absorber with a packed bed column was modified into a bioreactor for microbial gas culture of C. necator. FIG. 9 shows the schematic diagram of the experimental set up. The reactor (3 L) was filled with 1.5 L of the same liquid medium as above. During the experiment, the aqueous solution was re-circulated through an external loop and dispersed into the top of a 20 cm packed bed column of glass beads. The bed provided the contact area of gas and liquid. The positive replacement pump in the re-circulation loop generates a flow rate of 5.3 L/min. A gas stream (70% H₂, 20% O₂ and 10% CO₂) of 1.5 L/min was introduced to the reactor from the top gas inlet and discharged after contact with the concurrent liquid through the exhaust gas outlet. The exact concentrations of CO₂ and O₂ in the exhaust gas were monitored using a gas analyzer. After inoculated with 100 mL of seed solution, the same growth conditions as in the conventional bioreactor were maintained. Liquid samples were collected with time for OD, cell density, and PHB content.

FIG. 10 shows the variation of OD with the fermentation time for the conventional gas absorber. The highest OD of 14.0 was observed during the 90 h fermentation period. After a lag phase of 12 hours, the cells exhibited an exponential growth in the first 25 hours at a maximum specific growth rate (μₘₐₓ) of 0.13 h⁻¹. The specific growth rate was then gradually reduced to 0.03 h⁻¹ to give a linear increase of cell density, an indication of mass transfer limitation. The maximum cell density was around 4.2 g/L at a corresponding OD of 14 after a fermentation period of 90 h.

Performance of a Spray Nozzle Bioreactor According to the Present Invention (SNBR)

In the conventional bioreactors and gas absorbers, the gas is dispersed into a continuous phase of liquid. Because of poor gas solubility, the gas quickly forms bubbles and leaves the liquid solution, resulting in a short contact time for mass transfer. In order to maintain a high gas holdup in the water solution, a high gas flow rate is needed, which results in high gas wastage. In order to address these drawbacks, the bioreactor of the present invention is characterized by a reversed liquid-in-gas distribution as shown in FIG. 1. The medium solution 11 is circulated through an external loop 6 at 3.1 L/min and sprayed as tiny droplets into the gas phase through a spray nozzle 12 (0.18 cm orifice, Cole Parmer, Vernon Hills, Ill.). The gaseous substrate 14 was introduced into the overhead gas phase 15 present in the bioreactor at a desired flow rate through the conduit 25. Because the gas/liquid contact is determined by the liquid droplets, not by the gas holdup in the solution, the gas stream can be controlled at a very low flow rate depending on how fast the gases can be used by the microbial cells. More importantly, a high mass transfer rate (k_{L}a) can be maintained regardless of the gas flow rate. The retention time of the gas stream can also be adjusted by changing the volume of overhead space of the bioreactor. In this demonstration case, the spray nozzle is located at 10 cm above the liquid surface.

Similar to the experiments of conventional bioreactor and gas absorber above, the same liquid solution of 1.5 L was used in the nozzle spray bioreactor (3 L in total volume). After inoculated with 100 mL seed solution, the cells were grown on a gas stream of 0.45 L/min. The pH and temperature were maintained at 6.9 and 30.0° C., respectively. The pressure drop across the nozzle was measured using a digital pressure gauge (Cole Parmer, Vernon Hills, Ill.). Similar to previous experiments, liquid samples were analyzed for OD, cell density, and PHB content. Components of the exhaust gas were monitored by the gas analyzer.

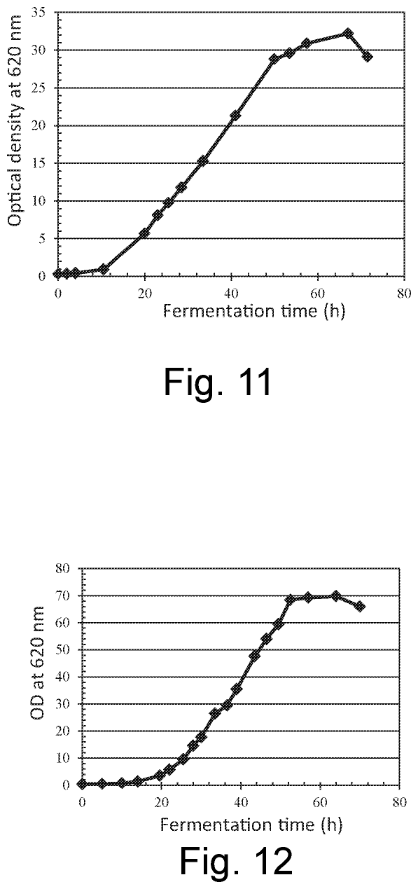

FIG. 11 shows cell growth or increase of the optical density with fermentation time. The highest OD of 32.2 was recorded after 72 h of fermentation. The corresponding cell density was 12.7 g/L. The maximum specific growth rate (μₘₐₓ) of 0.15 h⁻¹ was observed during the first 24 h period of the fermentation and then it was gradually reduced to 0.04 h⁻¹, indicating a possible gas mass transfer limitation. The pressure drop across the nozzle was maintained between 30.6 to 30.9 psi.

The volumetric mass transfer coefficient (k_{L}a) of oxygen was measured with an air stream at different flow rates. Specifically, the highest k_{L}a of 230 h⁻¹ was observed at air flow rate 0.75 L/min. Compared to the conventional bioreactor (Table 1), this is a significant improvement in terms of saving gas and reduce energy consumption.

Performance of Spray Nozzle Static Mixer Bioreactor (SNSMBR)

The spray nozzle bioreactor above was further improved by adding static mixers 7 in the liquid circulation loop 6 and introducing the gas stream 14 between the pump 4 and the mixers 7 as shown in FIG. 1. The gas and liquid streams were premixed in the static mixers (two in this case) and their contact was significantly enhanced under a high shear stress in the nozzle. The fine droplets of the liquid-gas mixture increase the gas mass transfer rate significantly. The k_{L}a was increased to 377 h⁻¹ at a low air flow rate of 0.75 L/min. Compared to the spray nozzle reactor (SNBR) of FIG. 1, the SNSMBR of FIG. 2 exhibits a 60% increase at the same gas flow rate. By comparison of the k_{L}a data of the conventional bioreactor (Table 1), the SNSMBR gives a 150% increase of k_{L}a (0.75 L/min air flow rate and 900 rpm agitation).

The same microbial culture of the experiment carried out in the SNBR was tested in the SNSMBR bioreactor shown in FIG. 2. The operational parameters such as pH, and temperature were similar to the previous experiments above. The medium solution in the reactor was 1.5 L and the recirculation flow rate was 3.1 L/min to give a liquid retention time of 0.48 min. The inlet gas flow rate was maintained at between 0.2 to 0.3 L/min (gassing rate of 0.13-0.2 vvm; superficial gas velocity of 0.00019-0.00028 m/s) and the gas mixture composition was similar to the previous experiments (70% H₂, 20% O₂ and 10% CO₂). Liquid samples were analyzed for OD, cell density, and PHB content. The composition of the exhaust gas was monitored by a gas analyzer.

FIG. 12 shows the time course of cell growth or optical density increase with the fermentation time in the SNSMBR. After being inoculated with 100 mL seed solution, the optical density of the fermentation broth was increased exponentially at a specific growth rate of 0.16 h⁻¹. The highest OD of 70 was reached in 65 h of fermentation. The corresponding cell density was 26.74 g/L and the final PHB content of the dry cell mass was 52%. The most significant achievement of the SNSMBR reactor system was the use of a very low gas flow rate (<0.3 L/min). Therefore, compared to the conventional bioreactor, gas wastage was significantly reduced. This saves H₂ gas which is considered as the most expensive feedstock in PHB production from the gaseous substrates, especially when the flue gas containing CO₂ and O₂ is used as the feedstock.

Comparison of Bioreactor Performance in Microbial Gas Cultures

Table 6 is a comparison of the two conventional bioreactors (CBR and CGA) described above and two bioreactors according to the present invention (SNBR and SNSMBR).

TABLE 6
Bioreactor performance in microbial gas cultures

| | | | Spray | Spray nozzle |
|---|---|---|---|---|
| | Conventional | Conventional | nozzle | with static |
| | bioreactor | gas absorber | bioreactor | mixers |
| Reactor type | (CBR) | (CGA) | (SNBR) | (SNSMBR) |
| Maximum OD | 72 | 14 | 32 | 70 |
| Maximum cell | 24.1 | 4.7 | 12.4 | 26.7 |
| density (g/L) | | | | |
| Agitation speed | 900 | n/a | n/a | n/a |
| (rpm) | | | | |
| PHB content | 59 | 56 | 53 | 52 |
| (% dry basis) | | | | |
| Gas flow rate | 1.5 | 1.5 | 0.5 | 0.2-0.3 |
| (L/min)^{a} | | | | |
| Liquid flow | n/a | 5.3 | 3.1 | 3.1 |
| rate (L/min) | | | | |
| Maximum k_{L}a | 144 | 230 | 280 | 377 |
| (h⁻¹) | | | | |
| Cell productivity | 0.23 | 0.047 | 0.18 | 0.41 |
| (g/L · hr) | | | | |
| Cell yield on H₂ | 0.068 | 0.014 | 0.16 | 0.73 |
| (g DCM/g H2)^{b} | | | | |

^{a}Gas flow rate at 1 atm, 30° C.
^{b}Gram of dry cell mass produced per gram H2

introduced into the bioreactors.

Compared to the conventional bioreactor (CBR), the SNSMBR gives a much higher cell productivity and PHB productivity, yet uses much less hydrogen. The dry cell mass yield based on the amount of hydrogen fed into the bioreactor is increased by more than 10 times.

1. A process for producing at least one polyhydroxyalkanoate through gas fermentation, the process comprising:
a) providing at least one gas fermentation vessel having an internal volume partially filled with a liquid fermentation broth that comprises:
water,
suspended gas-fermenting microorganisms capable of producing the at least one polyhydroxyalkanoate, and
nutrients for the gas-fermenting microorganisms,
a remaining part of the internal volume of the at least one gas fermentation vessel being filled with a gas phase;
b) continuously withdrawing an aliquot of the liquid fermentation broth from the at least one gas fermentation vessel;
c) supplying a gaseous substrate comprising CO₂, H₂, and O₂;
d) mixing the gaseous substrate with the liquid fermentation broth withdrawn from the at least one gas fermentation vessel by contacting the liquid fermentation broth in a form of sprayed droplets with the gaseous substrate in the gas phase;
e) cultivating the gas-fermenting microorganisms with a gas-liquid mixture obtained in step d) to form a cell mass containing the at least one polyhydroxyalkanoate; and
f) recovering the at least one polyhydroxyalkanoate from the cell mass.

2. The process of claim 1, comprising:
a) providing at least one gas fermentation vessel having an internal volume partially filled with a liquid fermentation broth that comprises:
water,
suspended gas-fermenting microorganisms capable of producing the at least one polyhydroxyalkanoate, and
nutrients for the gas-fermenting microorganisms;
a remaining part of the internal volume of the at least one gas fermentation vessel being filled with a gas phase;
b) continuously withdrawing an aliquot of the liquid fermentation broth from the at least one gas fermentation vessel;
c) supplying a gaseous substrate comprising CO₂, H₂, and O₂ and mixing the gaseous substrate with the liquid fermentation broth withdrawn from the at least one gas fermentation vessel;
d) spraying a so-obtained gas-liquid mixture into the at least one gas fermentation vessel and cultivating the gas-fermenting microorganisms to form a cell mass containing the at least one polyhydroxyalkanoate; and
e) recovering the at least one polyhydroxyalkanoate from the cell mass.

3. The process of claim 1, comprising:
a) providing at least one gas fermentation vessel having an internal volume partially filled with a liquid fermentation broth that comprises:
water,
suspended gas-fermenting microorganisms capable of producing the at least one polyhydroxyalkanoate, and
nutrients for the gas-fermenting microorganisms,
a remaining part of the internal volume of the at least one gas fermentation vessel being filled with a gas phase;
b) continuously withdrawing an aliquot of the liquid fermentation broth from the at least one gas fermentation vessel;
c) supplying a gaseous substrate comprising CO₂, H₂, and O₂ into the gas phase;
d) spraying the liquid fermentation broth withdrawn from the at least one gas fermentation vessel into the gas phase;
e) cultivating the gas-fermenting microorganisms with a gas-liquid mixture obtained in step d to form a cell mass containing the at least one polyhydroxyalkanoate; and
f) recovering the at least one polyhydroxyalkanoate from the cell mass.

4. The process of claim 1, wherein the liquid fermentation broth is withdrawn and re-introduced into the at least one gas fermentation vessel at a circulation rate greater than or equal to 2 liters per minute (l/min).

5. The process of claim 1, wherein the gaseous substrate is supplied into the at least one gas fermentation vessel with a gassing rate less than or equal to 0.001 meters per second (m/s).

6. The process of claim 4, wherein the gaseous substrate is supplied into the at least one gas fermentation vessel with a gassing rate less than or equal to 0.001 meters per second (m/s).

7. The process of claim 1, wherein the spraying of the liquid fermentation broth, optionally mixed with the gaseous substrate, is carried out through one or more nozzles each having a pressure drop less than or equal to 1.0×10⁵ pascals (Pa).

8. The process of claim 2, wherein the gaseous substrate is mixed with the liquid fermentation broth withdrawn from the at least one gas fermentation vessel in at least one static mixer before being circulated and sprayed into the at least one gas fermentation vessel.

9. The process of claim 1, wherein the gaseous substrate comprises flue gas.

10. A gas fermentation bioreactor, comprising:
a vessel having an internal volume partly filled with a liquid fermentation broth that comprises gas-fermenting microorganisms and a fermentable medium, a remaining part of the internal volume of the vessel being filled with a gas phase;
at least one effluent circulation conduit communicating with an interior of the vessel through which an aliquot of the liquid fermentation broth is withdrawn, the at least one effluent circulation conduit being connected to a circulation line configured to circulate the aliquot of the liquid fermentation broth externally with respect to the vessel and then re-introducing the aliquot of the liquid fermentation broth into the gas phase present in the vessel;
at least one spraying nozzle, connected to the circulation line, configured to spray the liquid fermentation broth into the gas phase; and
a feeding system configured to feed into the gas phase a gaseous substrate comprising one or more gases to cultivate the gas-fermenting microorganisms;
wherein the feeding system is connected to the gas phase through an inlet conduit communicating with the interior of the vessel.

11. A gas fermentation bioreactor, comprising:
a vessel having an internal volume partly filled with a liquid fermentation broth that comprises gas-fermenting microorganisms and a fermentable medium, a remaining part of the internal volume of the vessel being filled with a gas phase;
at least one effluent circulation conduit communicating with an interior of the vessel through which an aliquot of the liquid fermentation broth is withdrawn, the at least one effluent circulation conduit being connected to a circulation line configured to circulate the aliquot of the liquid fermentation broth externally with respect to the vessel and then re-introducing the aliquot of the liquid fermentation broth into the gas phase present in the vessel;
at least one spraying nozzle, connected to the circulation line, configured to spray the liquid fermentation broth into the gas phase; and
a feeding system configured to feed, into the liquid fermentation broth withdrawn from the vessel, a gaseous substrate comprising one or more gases to cultivate the gas-fermenting microorganisms;
wherein the feeding system is connected to the circulation line so that a gas-liquid mixture so-obtained is sprayed into the gas phase present in the vessel by the at least one spraying nozzle.

12. The gas fermentation bioreactor of claim 11, wherein the circulation line comprises at least one static mixer configured to mix the liquid fermentation broth withdrawn from the vessel and the gaseous substrate to form the gas-liquid mixture.

13. The process of claim 1, wherein the liquid fermentation broth is withdrawn and re-introduced into the at least one gas fermentation vessel at a circulation rate greater than or equal to 3 liters per minute (l/min).

14. The process of claim 1, wherein the gaseous substrate is supplied into the at least one gas fermentation vessel with a gassing rate less than or equal to 0.0005 meters per second (m/s).

15. The process of claim 4, wherein the gaseous substrate is supplied into the at least one gas fermentation vessel with a gassing rate less than or equal to 0.0005 meters per second (m/s).

16. The process of claim 1, wherein the at least one polyhydroxyalkanoate comprises polyhydroxybutyrate.

17. The gas fermentation bioreactor of claim 10, wherein the cultivated gas-fermenting microorganisms form a cell mass containing at least one polyhydroxyalkanoate.

18. The gas fermentation bioreactor of claim 17, wherein the at least one polyhydroxyalkanoate comprises polyhydroxybutyrate.

19. The gas fermentation bioreactor of claim 11, wherein the cultivated gas-fermenting microorganisms form a cell mass containing at least one polyhydroxyalkanoate.

20. The gas fermentation bioreactor of claim 19, wherein the at least one polyhydroxyalkanoate comprises polyhydroxybutyrate.
